(19) United States
(12) Patent Application Publication
(10) Pub. No.: US 2014/0350430 A1
(43) Pub. Date: Nov. 27, 2014

(54) APPARATUS FOR TESTING RESPIRATORY FUNCTION

(71) Applicant: DENSO CORPORATION, Kariya-city (JP)

(72) Inventors: Rie OSAKI, Anjo-shi (JP); Shinji NANBA, Kariya-shi (JP); Mitsuyuki KOBAYASHI, Gamagori-shi (JP); Taiji KAWACHI, Kariya-shi (JP)

(73) Assignee: DENSO CORPORATION, Kariya-city (JP)

(21) Appl. No.: 14/284,851

(22) Filed: May 22, 2014

(30) Foreign Application Priority Data
May 24, 2013 (JP) 2013-109953

(51) Int. Cl. A61B 5/091

(52) U.S. Cl. CPC A61B 5/091
USPC 600/538

(57) ABSTRACT

A respiratory function testing apparatus capable of testing a respiratory function of a subject more accurately. In the apparatus, a respiratory state detection unit acquires a first signal representative of different inspiratory volumes corresponding to a plurality of breaths of the subject and a second signal representative of intrapleural pressures corresponding to the respective different inspiratory volumes, and detects a plurality of respiratory states corresponding to the different inspiratory volumes and their corresponding intrapleural pressures. A respiratory state determination unit captures a state of the respiratory function of the subject on the basis of the plurality of respiratory states corresponding to the different inspiratory volumes and their corresponding-respective intrapleural pressures.

### CROSS-REFERENCE TO RELATED APPLICATION

This application is based on and claims the benefit of priority from earlier Japanese Patent Application No. 2013-109953 filed May 24, 2013, the description of which is incorporated herein by reference.

### BACKGROUND

1. Technical Field

The present invention relates to techniques for testing a respiratory function of a subject, such as lung compliance or the like.

2. Related Art

In recent years, lung diseases, such as pneumonia, chronic obstructive pulmonary disease (COPD) and the like, are increasing all over the world. The lung compliance representative of softness of the lung is said to be a useful indicator for screening and/or determining therapeutic efficacy for lung diseases. To measure such lung compliance, it is required to measure an intrapleural pressure. However, it is difficult to measure the intrapleural pressure. Instead of the intrapleural pressure, an esophageal pressure may be measured. To measure the esophageal pressure, however, it is required to insert a balloon catheter into the esophagus, which may cause great discomfort for a patient. Therefore, the measurement of the lung compliance cannot be performed with ease.

To overcome such disadvantages, known techniques, such as disclosed in Japanese Patent Application Laid-Open Publication No. 2010-142594, use a blood pressure transducer for measuring a blood pressure (invasive blood pressure) and electrocardiogram electrodes for measuring a heartbeat period to extract a respiratory function signal representative of respiratory function from a blood pressure waveform signal detected by the blood pressure transducer with use of an electrocardiographic waveform signal caused by the heart contraction, acquired from the electrocardiogram electrodes.

The prior art techniques set forth above can reduce the burden of measuring the esophageal pressure, but has a disadvantage that the respiratory function cannot be tested accurately.

In consideration of the foregoing, exemplary embodiments of the present invention are directed to providing techniques capable of testing the respiratory function more accurately.

### SUMMARY

In accordance with an exemplary embodiment of the present invention, there is provided an apparatus for testing a respiratory function of a subject. The apparatus includes a respiratory state detection unit which acquires a first signal (such as an inspiratory signal) representative of different inspiratory volumes corresponding to a plurality of breaths of the subject and a second signal (such as a signal acquired from a pulse wave signal) representative of intrapleural pressures corresponding to the respective different inspiratory volumes, and detects a plurality of respiratory states (such as information about coordinate points representative of the respective different inspiratory volumes and the respective intrapleural pressures) corresponding to the different inspiratory volumes and their corresponding intrapleural pressures. The apparatus further includes a respiratory state determination unit which captures a state of the respiratory function of the subject on the basis of the plurality of respiratory states corresponding to the different inspiratory volumes and their corresponding intrapleural pressures.

According to the studies by the present inventors, as described later, it has been found that, for a specific subject, there is a linear relationship as expressed by a specific relationship (specifically, expressed by a first order equation) between the inspiratory volume for each inspiration and its corresponding intrapleural pressure.

Using the first order equation to express such a relationship, it has been established that a slope of the first order equation corresponds to the expansibility of the lungs (i.e., the lung compliance) and an intercept of the first order equation corresponds to the expirability of the lungs or the % volume of air in the lungs which is expirable.

With this configuration, the data on the plurality of respiratory states representative of the relationship between the inspiratory volume for each inspiration and its corresponding intrapleural pressure allows a state of the subject's respiratory function to be detected accurately.

### BRIEF DESCRIPTION OF THE DRAWINGS

In the accompanying drawings:

FIG. 1 shows a schematic block diagram of a respiratory function testing system including a respiratory function testing apparatus in accordance with one embodiment of the present invention;

FIGS. 2A-2B show graphs of inspiratory volume versus estimated intrapleural pressure for first and second cases, respectively;

FIG. 3 shows a graph of estimated intrapleural pressure versus estimated lung compliance at end-expiration;

FIG. 4 shows a flowchart of a process of determining a slope and an intercept of a first order equation representative of respiratory function from respiratory data and pulse wave data;

FIG. 5 shows a graph of inspiratory volume versus a number of breaths;

FIG. 6 shows a flowchart of a process of estimating an intrapleural pressure from pulses;

FIG. 7 shows a graph of a pulse wave signal waveform;

FIG. 8 shows a graph of a pulse wave signal and envelopes;

FIG. 9 shows a graph representative of correlations between intrapleural pressures and measured values of esophageal pressure based on pulse waves;

FIG. 10A shows a graph representative of a relationship between first and second envelopes;

FIG. 10B shows a graph representative of an intrapleural pressure signal;

FIG. 10C shows a graph representative of a pressure in a mouthpiece;

FIG. 11 shows an example of system configuration during calibration; and

FIG. 12 shows a modification for changing a respiratory state.

### DESCRIPTION OF THE PREFERRED EMBODIMENTS

The present invention will be described more fully hereinafter with reference to the accompanying drawings. Like numbers refer to like elements throughout.

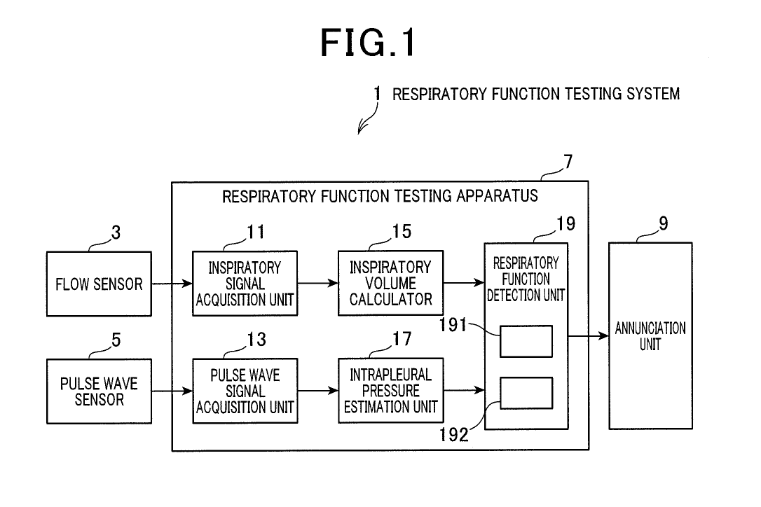

a) A respiratory function testing system including a respiratory function testing apparatus in accordance with one embodiment of the present invention will now be explained with reference to FIG. 1. This respiratory function testing system 1, as described later, is configured to test a respiratory function of a subject on the basis of data of inspiratory volumes as a subject breathes and data of intrapleural pressures acquired from pulse waves (according to an intrapleural pressure estimating method).

As shown in FIG. 1, the respiratory function testing system 1 includes an flow sensor 3 configured to detect a gas flow rate (inspiratory flow) as the subject inspires, a pulse wave sensor 5 configured to detect a pulse wave of the subject, a respiratory function testing apparatus 7 configured to test a respiratory function on the basis of an inspiratory signal representative of an inspiratory flow from the flow sensor 3 and a pulse wave signal representative of the pulse wave from the pulse wave sensor 5, and an annunciation unit 9 configured to output a test result from the respiratory function testing apparatus 7.

The flow sensor 3 includes, but is not limited to, a well-known differential pressure based or hot-wire based flow sensor capable of detecting a gas flow rate. The flow sensor 3 outputs an electrical signal representative of an inspiratory flow to the respiratory function testing apparatus 7.

The pulse wave sensor 5, which may be an optical based sensor including a well-known light emitting device (LED) and a well-known photosensitive device (PD), is configured to detect a pulse wave (volume pulse wave), for example, by irradiating a fingertip of the subject and receiving the reflected light. The pulse wave sensor 5 outputs a pulse wave signal representative of a state of the pulse wave to the respiratory function testing apparatus 7.

The respiratory function testing apparatus 7, which may be an electronic control unit (ECU) formed of a well-known microcomputer as a main component, is configured to test the respiratory function and control the annunciation unit 9 on the basis of the inspiratory signal from the flow sensor 3 and the pulse wave signal from the pulse wave sensor 5.

The annunciation unit 9 includes a display, such as a liquid crystal display or the like, and a speaker, to annunciate a test result for the respiratory function acquired from the respiratory function testing apparatus 7. Functions of the respiratory function testing apparatus 7 will be explained in more detail.

As shown in FIG. 1, the respiratory function testing apparatus 7 includes an inspiratory signal acquisition unit 11, a pulse wave signal acquisition unit 13, an inspiratory volume calculation unit 15, an intrapleural pressure estimation unit 17, and a respiratory function detection unit 19.

The inspiratory signal acquisition unit 11 is configured to acquire an inspiratory signal representative of an inspiratory volume per unit time (i.e., a gas flow rate) from the flow sensor 3. The pulse wave signal acquisition unit 13 is configured to drive the pulse wave sensor 5 to acquire a pulse wave signal representative of a pulsation state of the blood vessel.

The inspiratory volume calculation unit 15 is configured to calculate an inspiratory volume for each inspiration of the subject on the basis of the inspiratory signal. More specifically, the inspiratory volume calculation unit 15 acquires the inspiratory volume by integrating the inspiratory flow (i.e., the inspiratory volume per unit time) acquired from the inspiratory signal. The intrapleural pressure estimation unit 17, as described later, is configured to estimate an intrapleural pressure by analyzing the pulse wave signal.

The respiratory function detection unit 19, as described later, is configured to test or determine the respiratory function on the basis of data of the inspiratory volume calculated by the inspiratory volume calculator 15 and the intrapleural pressure estimated by the intrapleural pressure estimation unit 17.

b) The principle of testing the respiratory function in the respiratory function testing apparatus 7 will now be explained. According to the studies by the present inventors, it has been found that, for a specific subject, there is a linear relationship as expressed by a first order equation (y=ax+b) between the inspiratory volume for each inspiration and its corresponding intrapleural pressure (for example, at end-inspiration). The variables y, x represent the inspiratory volume (V) and the (estimated) intrapleural pressure (P), respectively.

It has been established that a slope a (Δ(V/P)) of the first order equation corresponds to the expansibility of the lungs (i.e., the lung compliance) and an intercept b (X-intercept) of the first order equation corresponds to the expirability.

In particular, the establishment that the X-intercept corresponds to the expirability of the lungs comes from an experimental result that the X-intercept and the expiratory resistance are correlated (with the determination coefficient R2=0.84). Hence in the present embodiment, for a plurality of respirations (inspirations) conducted at predetermined time intervals with different inspiratory volumes, for example, a shallow breath (K1), a normal breath (K2) and a deep breath (K3) conducted at predetermined time intervals with respective different inspiratory volumes K1-K3 (where K1<K2<K3), the inspiratory volume and the intrapleural pressure for each inspiration are plotted to a Cartesian coordinate point (X, Y), where the X- and Y-coordinates represent the inspiratory volume and the intrapleural pressure, respectively.

The first order equation is derived from the plurality of plotted coordinate points. Then, from the first order equation, the slope a and the intercept b of the first order equation are acquired. The respiratory function is determined from the slope a and the intercept b of the first order equation. It should be noted that, since at least two coordinate points are needed to determine the first order equation, at least two inspirations have to be conducted for a specific subject. For more than two coordinate points, the first order equation may be determined, for example, as an approximate line (referred to as a regression line) determined by a well-known least-square technique.

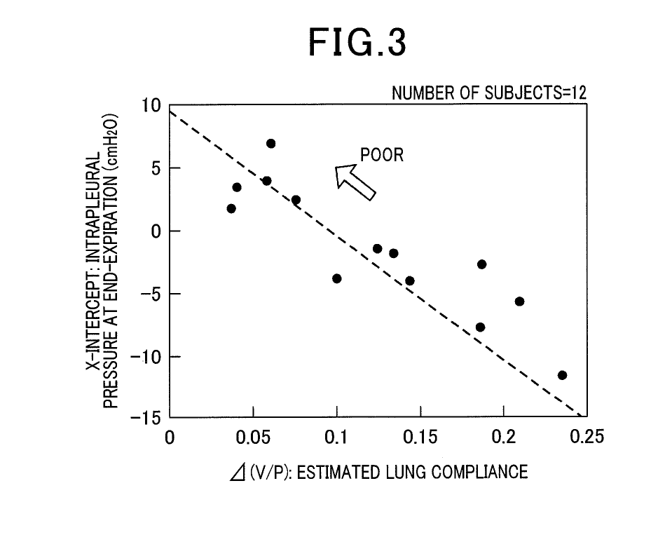

FIG. 3 is a graph showing data for a plurality of subjects (for example, twelve subjects) acquired as above, where the vertical axis represents the intercept b (X-intercept: intrapleural pressure at end-expiration) and the horizontal axis represents the slope value a (ΔV/P: estimated lung compliance).

As can be seen from this graph, when the X-intercept representative of the intrapleural pressure is high and the slope a representative of the lung compliance is low (e.g., although the X-intercept representative of the intrapleural pressure is high), the respiratory function may be assumed to be poor.

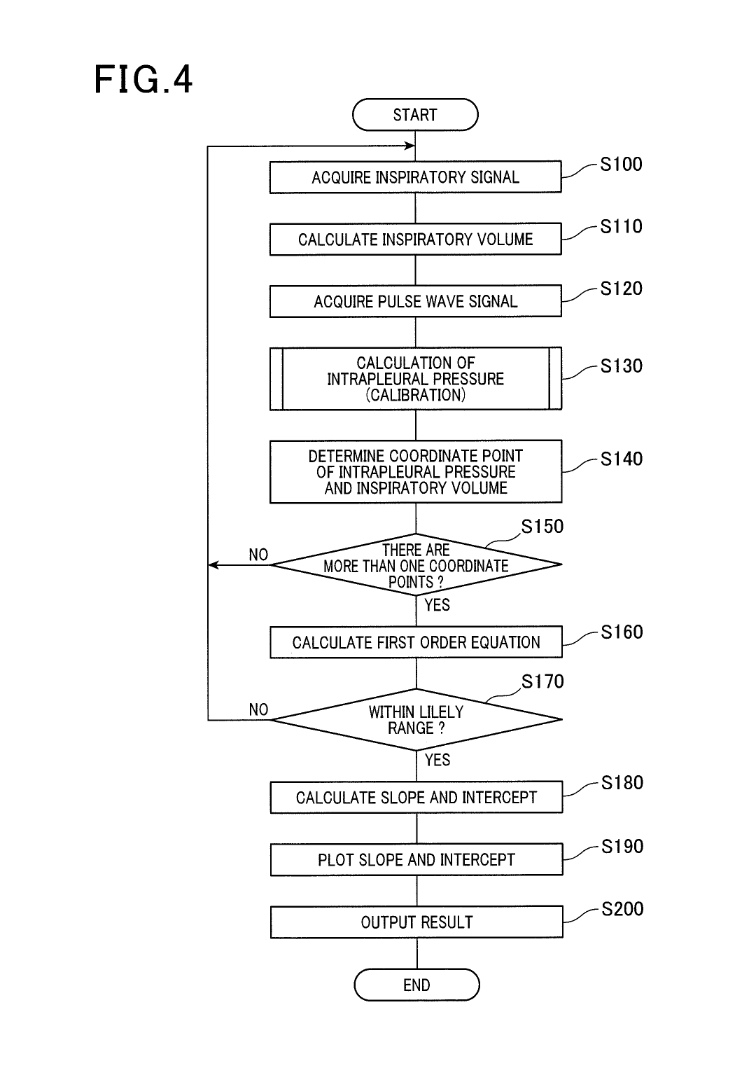

c) A process of testing the respiratory function performed in the respiratory function testing apparatus 7 on the basis of the principle set forth above will now be explained with reference to FIG. 4 and other drawings.

<1> Main Process

As shown in FIG. 4, in step S100, the inspiratory signal is acquired from the flow sensor 3.

Subsequently, in step S110, the acquired inspiratory signal is integrated to acquire an inspiratory volume corresponding to the integrated value of the inspiratory signal. That is, since the inspiratory signal is representative an inspiratory volume per unit time (referred to as an inspiratory flow), integration of the inspiratory signal leads to an inspiratory volume. With a device capable of directly measuring the inspiratory volume, data on the inspiratory volume may be acquired from such a device.

In particular, in the present embodiment, the respiratory function is tested on the basis of relationships between inspiratory volumes in a plurality of different respiratory states (inspiratory states) and their respective intrapleural pressures, so it is necessary to acquire a plurality of different inspiratory volumes in the respective respiratory states.

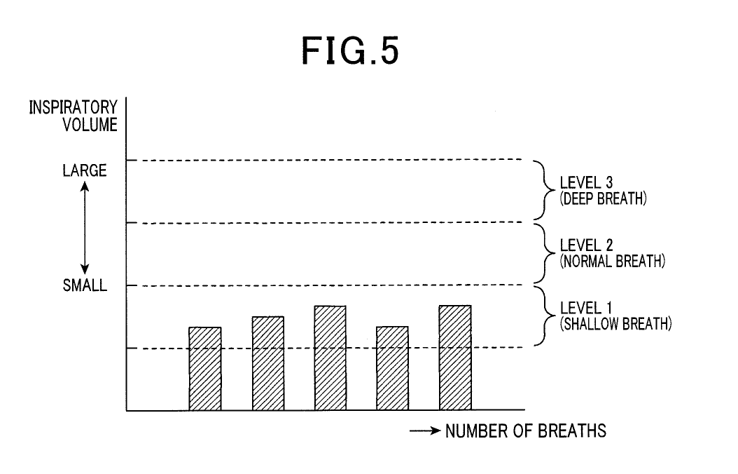

The plurality of different respiratory states may be acquired, for example, by asking the subject to breathe shallowly, normally and deeply and acquiring inspiratory volumes in the respective breaths. For the subject, however, it is difficult to discriminate between the respiratory states. Preferably, as shown in FIG. 5, the annunciation unit 9 may be configured to display a graph of the calculated inspiratory volumes to discriminate between the shallow breathing, the normal breathing and the deep breathing. Alternatively, the annunciation unit 9 may be configured to show respiratory state levels, e.g., a higher level, a normal level or a lower level, by voice or light and other means to show what level of the respiratory state is.

Since measuring the inspiratory volume only once for each respiratory state may lead to an error, it is desirable to, for each respiratory state, measure the inspiratory volume multiple times and use an average over the inspiratory volumes measured for the respiratory state. Subsequently, in step S120, a pulse wave signal is acquired from the pulse wave sensor 5.

More specifically, a sensor output of the pulse wave sensor 5 is fed to the respiratory function testing apparatus 7 and amplified therein to acquire an analog signal. Thereafter the analog signal is converted into a digital signal to be fed to the microcomputer.

In step S130, an intrapleural pressure is estimated from the pulse wave signal in a manner as described later. Alternatively, the operations in steps S100, S110 may be preceded by the operations in steps S120, S130. Still alternatively, the operations in steps S100, S110 may be performed in parallel with the operations in steps S120, S130.

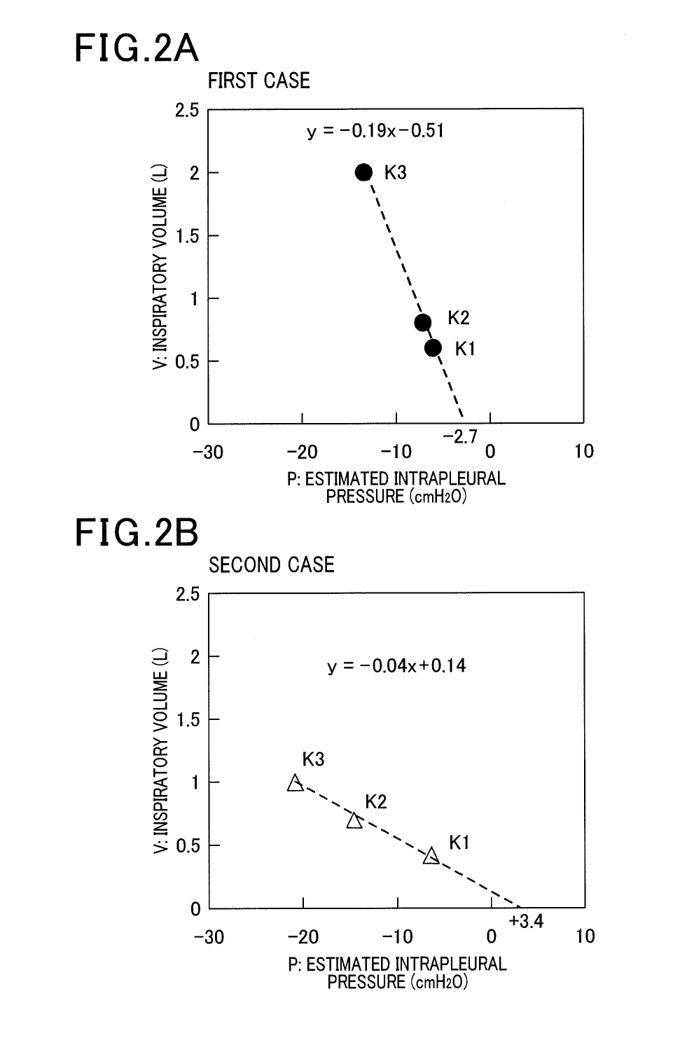

In step S140, as shown in FIG. 2, a coordinate point (X, Y) for each inspiratory action acquired as above is plotted in the XY-Cartesian coordinate system, where the X-axis is the intrapleural pressure and the Y-axis is the inspiratory volume.

For example, as shown in FIG. 2A, a coordinate point for the shallow breathing K1 is determined by the inspiratory volume and the intrapleural pressure calculated for the shallow breathing K1. When the average of inspiratory volumes over multiple shallow breaths is used for determining the coordinate point, an average of intrapleural pressures over the multiple shallow breaths may be used for the corresponding coordinate point.

In step S150, it is determined whether or not there are more than one coordinate points for different respiratory states (i.e., more than one different inspiratory volumes). If it is determined that there are more than one coordinate points for different respiratory states, then the process proceeds to step S160. If it is determined that there is only one or no coordinate point, then the process returns to step S100 and then the similar operations as set forth above are repeated.

In step S160, as shown in FIG. 2A, a first order equation is acquired that connects the one or more coordinate points. When there are two or more coordinate points as shown in FIG. 2B, the first order equation that best fits the distribution of the coordinate points (i.e., an approximate line) may be acquired according to the method of least squares.

In step S170, it is determined whether or not the first order equation acquired in step S160 is within a likely range such that a first order equation within the likely range is likely to represent the respiratory function correctly. If it is determined that the first order equation acquired in step S160 is within the likely range, then the process proceeds to step S180. If it is determined that the first order equation acquired in step S160 is out of the likely range, then the process returns to step S100 and then the similar operations as set forth above are repeated.

For example, the likely range for the first order equation likely to represent the respiratory function correctly may be predefined by experiments. When the first order equation acquired as above is out of such a likely range, it may be assumed that there are some erroneous measurements, so that the first order equation is inhibited from being used.

In step S180 after the first order equation is determined to correctly represent the respiratory function, a slope and an intercept b of the first order equation are calculated. In step S190, as shown in FIG. 3, the intercept b (X-intercept: intrapleural pressure at end-expiration) and the slope a (Δ(V/P):estimated lung compliance) are plotted in a graph on the XY-coordinate plane.

Hence, the position of the coordinate point plotted as above (see FIG. 3, where the coordinate points for twelve subjects are plotted) allows the respiratory function to be determined. For example, as the coordinate point is positioned toward the upper left of the graph, the respiratory function is considered poorer (or less desirable). The respiratory function can thus be determined on the basis of where the coordinate point is positioned on the XY-coordinate plane.

In step S200, the plotted result is displayed on the display of the annunciation unit 9. Alternatively or additionally, the diagnostic outcome of the respiratory function determined from the position of the coordinate point plotted as above may be displayed. Thereafter, the process ends.

<2> Process of Estimating Intrapleural Pressure

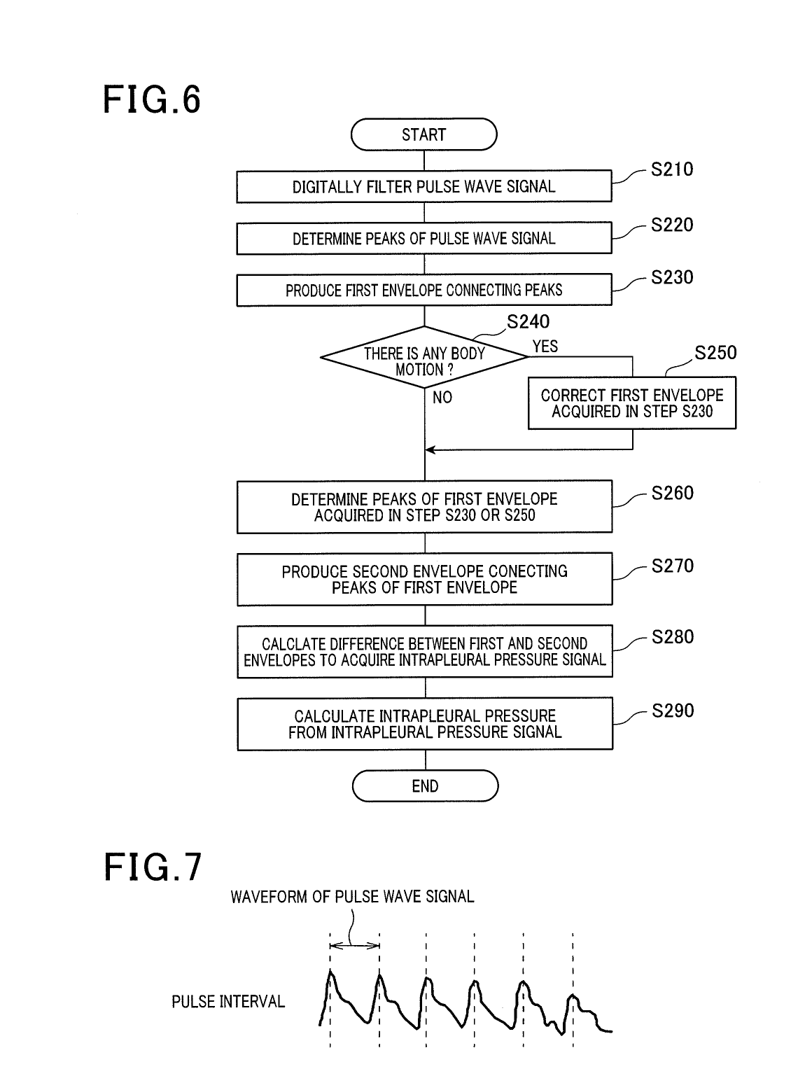

A process of estimating the intrapleural pressure from the pulse wave signal performed in step S130 will now be explained with reference to FIG. 6. This process is similar as in disclosed in Japanese Patent Application Laid-Open Publication No. 2002-355227.

As shown in FIG. 6, in step S210, the pulse wave signal is digitally filtered to extract an intrapleural pressure signal from the pulse wave signal. In this digitally filtering process to extract the intrapleural pressure signal from the pulse wave signal from a digital signal, noise of equal to or higher than 3 Hz, such as extraneous light noise, and signals of equal to or lower than 0.1 Hz (lower than the frequency of the intrathoracic signal) caused by body motion are removed from the digital signal.

In the following steps, a process of extracting features of the waveform of the pulse wave signal acquired in step S210 to quantify the pulse wave signal is performed, where features of the waveform of the pulse wave signal are extracted by using fluctuations or variations of the pulse wave signal.

More specifically, in step S220, peaks are determined for respective pulse waves as shown in FIG. 7. FIG. 7 shows a change over time of a signal output (voltage) of the pulse wave signal, where the vertical axis is the magnitude in volts (V) of the pulse wave signal relative to a reference value of 0[V].

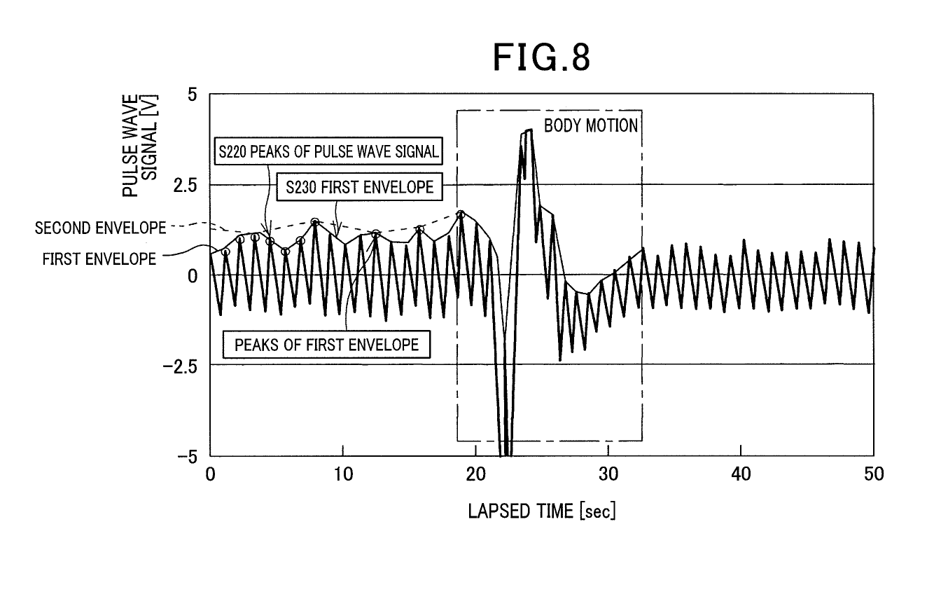

In step S230, a first envelope (represented by a thin line in FIG. 8) is produced by connecting the peaks acquired in step S220. In step S240, it is determined whether or not there is any body motion according to a well-known body motion determination method as disclosed in Japanese Patent Application Laid-Open Publication No. 2002-355227. If it is determined that there is any body motion, then the process proceeds to step S250. If there is no body motion, then the process proceeds to step S260.

In step S250, to remove the effects of the body motion from the first envelope acquired in step S230, the first envelope is corrected in a well-known envelope correcting method as disclosed in Japanese Patent Application Laid-Open Publication No. 2002-355227 after completion of the body motion.

In step S260, peaks of the first envelope acquired in step S230 or in step S250 are determined.

In step S270, a second envelope (represented by a dashed line in FIG. 8) is produced by connecting the peaks of the first envelope acquired in step S230 or in step S250. In step S280, an intrapleural pressure signal is determined as a difference between the first and second envelopes.

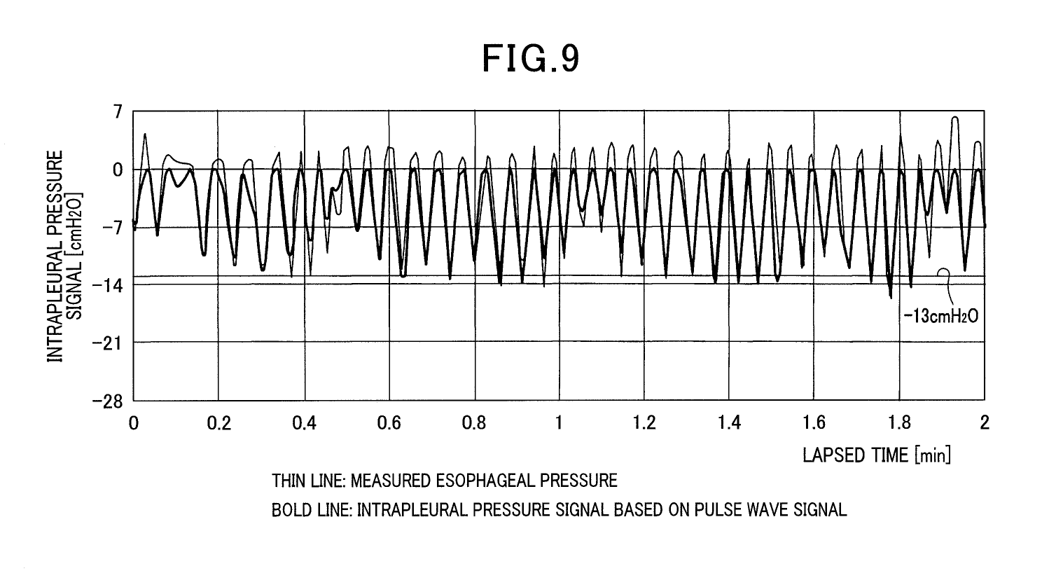

More specifically, studies by the inventors as disclosed in Japanese Patent Application Laid-Open Publication No. 2002-355227 have shown that the difference between the first and second envelopes is strongly correlated with the measured value of esophageal pressure representative of the actual intrapleural pressure (see FIG. 9). Hence the difference between the first and second envelopes may be determined as a signal representative of the intrapleural pressure (intrapleural pressure signal).

As shown in FIG. 9, the intrapleural pressure signal varies with respiratory actions. Hence, for example, a trough of the intrapleural pressure signal for each respiratory (inspiratory)action, where the intrapleural pressure reaches a maximum negative pressure, may be used as the intrapleural pressure signal representative of the intrapleural pressure.

In step S290, the intrapleural pressure (absolute value) is calculated from the intrapleural pressure signal through calibration as described later. Thereafter, the process ends.

d) There will now be explained the calibration for calculating the intrapleural pressure.

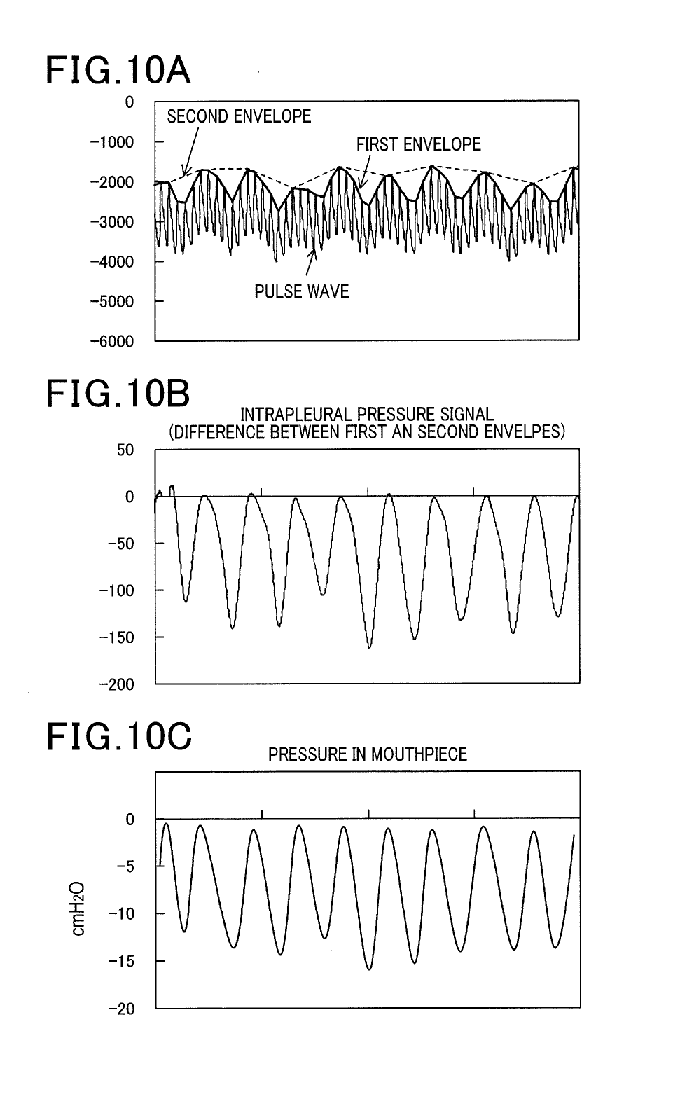

As shown in FIGS. 10A-10B, in the present embodiment, the intrapleural pressure signal is determined as a difference between the first and second envelopes. However, the intrapleural pressure signal takes a relative value (that is a value of the first envelope relative to a value of the second envelope), so it is necessary to estimate an absolute value of the intrapleural pressure.

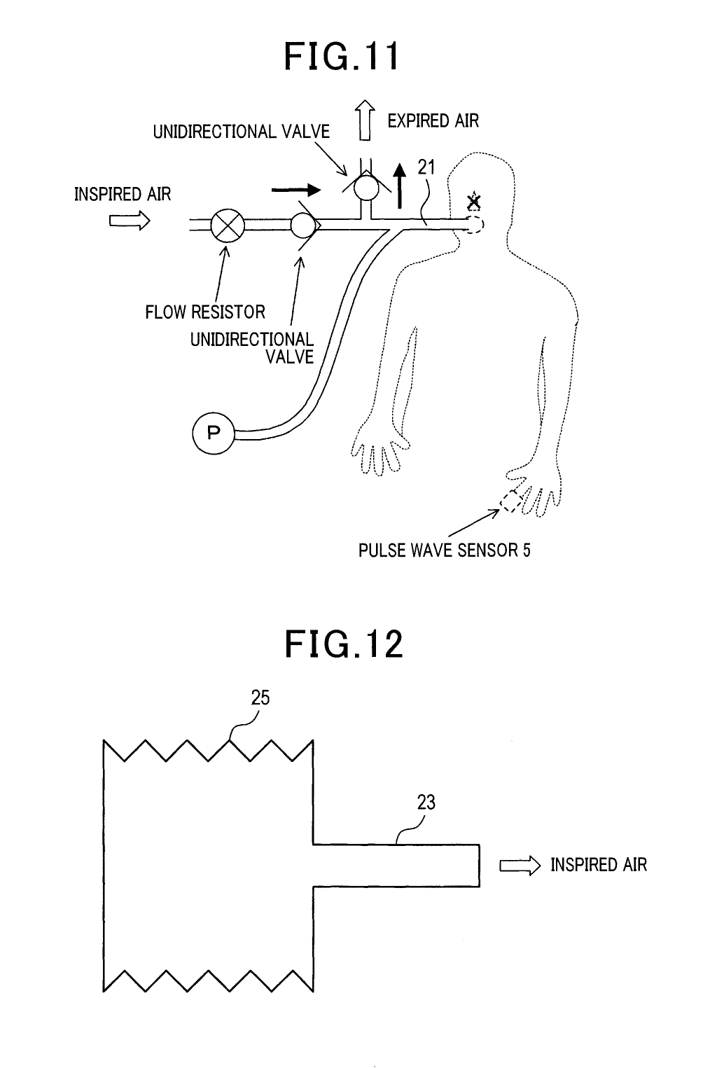

More specifically, there is a need to calculate a conversion factor for each subject, which represents what amount of change in intrapleural pressure an amount of relative change in the intrapleural pressure signal corresponds to. To this end, as shown in FIG. 11, each subject uses a nose clip and a mouthpiece attached to the face of the subject and is asked to breathe (deeply). As the subject breathes (deeply), a pressure in the mouthpiece of the subject is measured (see FIG. 10C). The calibration is conducted with use of the measured pressure in the mouthpiece.

Referring to FIG. 11, the flow resistor is configured such that the pressure in the mouthpiece P falls within a range of 20 cmH₂O-−30 cmH₂O (regardless of the inspiratory volume) during the calibration when the subject breathes deeply.

As can be seen from the FIGS. 10B-10C, the intrapleural pressure signal and the pressure in the mouthpiece are strongly correlated with each other. Therefore, a conversion factor for converting the intrapleural pressure signal into the absolute value such as the pressure in the mouthpiece can be known.

Therefore, the absolute value of the intrapleural pressure can be calculated or derived from the intrapleural pressure signal with use of the conversion factor. In the calibration, the intrapleural pressure signal is normalized by an average wave height of the pulse wave signal. That is, when the pulse wave signal may vary in magnitude due to variation in pressing pressure or the like for the pulse wave sensor 5, the intrapleural pressure signal may proportionally vary in magnitude, so it is necessary to correct the intrapleural pressure signal by dividing the intrapleural pressure signal by the average wave height of the pulse wave signal.

Referring again to FIG. 1, in the present embodiment, the respiratory function detection unit 19 includes a respiratory state detection unit (191, S100-S150) and a respiratory state determination unit (192, S160-S190).

The respiratory state detection unit 191 is configured to acquire the inspiratory signal (as a first signal) representative of different inspiratory volumes corresponding to a plurality of breaths of each subject and the pulse wave signal (as a second signal) presentative of the intrapleural pressures corresponding to the respective different inspiratory volumes, and detects the plurality of respiratory states (such as information about coordinate points representative of the respective different inspiratory volumes and the respective intrapleural pressures) corresponding to the different inspiratory volumes and their corresponding intrapleural pressures. The respiratory state determination unit 192 is configured to capture a state of the respiratory function of the subject on the basis of the plurality of respiratory states corresponding to the different inspiratory volumes and their corresponding-intrapleural pressures. As such, the respiratory state detection unit 191 is responsible for execution of the operations in steps S100-S150 (see FIG. 4). The respiratory state determination unit 192 is responsible for execution of the operations in steps S160-S190 (see FIG. 4). In particular, the respiratory state detection unit 191 is responsible for execution of the process of estimating the intrapleural pressure from the pulse wave signal (i.e., the calibration) in step S130 (see FIG. 6).

e) As above, in the present embodiment, the inspiratory volume is acquired from the inspiratory signal and the intrapleural pressure is estimated from the pulse wave signal. The coordinate point for each inspiratory action is plotted in the XY-coordinate plane, where the y-axis is the inspiratory volume and the x-axis is the intrapleural pressure. After a plurality of such coordinate points are plotted, a first order line (or an approximate line) connecting these coordinate points is determined. Thereafter, the slope a and the intercept b of the first order line are acquired.

The slope a and the intercept b represent the lung compliance and the expirability of the lungs, respectively. The respiratory function of the subject is determined from the magnitude of the slope a and the magnitude of the intercept b. For example, when the x-intercept representative of the intrapleural pressure is high and the slope a representative of the lung compliance is low (e.g., although the x-intercept representative of the intrapleural pressure is high), it may be determined that the respiratory function is poor.

It is to be understood that the invention is not to be limited to the specific embodiments disclosed above and that modifications and other embodiments are intended to be included within the scope of the appended claims. (1) For example, in the embodiments set forth above, the subject is asked to adjust the respiratory state (e.g., take shallow breaths or the like). Alternatively, there may be used a device for limiting the respiratory volume (volume of inspired air) to adjust the respiratory state.

For example, as shown in FIG. 12, a variable capacity container 25 may be attached to the distal end of the mouthpiece 23. More specifically, a small capacity container 25 allows the subject to breathe (inspire) shallowly. A large capacity container 25 allows the subject to breathe (inspire) deeply.

(2) In the embodiments set forth above, there has been described the respiratory function testing apparatus. The present invention can also be applied to a non-transitory computer-readable storage medium encoded with a computer program including instructions that, when executed by a data processing apparatus (e.g., the microcomputer), implement the algorithms set forth above.

The non-transitory computer-readable storage medium may include, but is not limited to, a storage medium of an electronic control unit (ECU) as the microcomputer, a microchip, a flexible disk unit, a hard disk, an optical disk or the like.

The program may include, but is not limited to a program stored in the digital storage medium, a program transmitted and received via a communication line, such as the Internet.

(3) The respiratory function testing apparatus may receive the signals directly from the pulse wave sensor and the pulse wave sensor. Alternatively, the respiratory function testing apparatus may receive the signals indirectly from the pulse wave sensor and the pulse wave sensor that are remote from the respiratory function testing apparatus, where data from the pulse wave sensor and the pulse wave sensor is stored in a personal computer (or in a digital storage medium) and the data is transmitted to the respiratory function testing apparatus remote from the pulse wave sensor and the pulse wave sensor via the Internet or the like to be used to test the respiratory function.

The signals acquired from the pulse wave sensor and the pulse wave sensor may be stored in a personal computer (or in a digital storage medium) for several days, and the signals may be used later to test or evaluate the respiratory function.

(4) In the present invention, the function of components in the embodiment set forth above may be distributed among a plurality of components, or functions of a plurality of components may be integrated in one component. At least part of the component(s) in the embodiment set forth above may be replaced with well-known component(s) having a similar function. Further, at least part of a component in the embodiment set forth above may be added to a component of other embodiments.

What is claimed is:

1. An apparatus for testing a respiratory function of a subject, comprising:
a respiratory state detection unit configured to acquire a first signal representative of different inspiratory volumes corresponding to a plurality of breaths of the subject and a second signal representative of intrapleural pressures corresponding to the respective different inspiratory volumes, and detect a plurality of respiratory states corresponding to the different inspiratory volumes and their corresponding intrapleural pressures: and
a respiratory state determination unit configured to capture a state of the respiratory function of the subject on the basis of the plurality of respiratory states corresponding to the different inspiratory volumes and their corresponding intrapleural pressures.

2. The apparatus of claim 1, wherein the respiratory state determination unit is configured to determine the state of the respiratory function of the subject on the basis of the plurality of respiratory states corresponding to the different inspiratory volumes and their corresponding-intrapleural pressures.

3. The apparatus of claim 1, wherein the respiratory state determination unit is configured to determine an equation which relates the inspiratory volumes to their corresponding-intrapleural pressures on the basis of the plurality of respiratory states.

4. The apparatus of claim 1, wherein the respiratory state detection unit is configured to plot XY-coordinate points for the respective respiratory states in an XY-coordinate plane with the Y- and X-coordinates for each coordinate point representing the inspiratory volume and the intrapleural pressure for the corresponding respiratory state, respectively.

5. The apparatus of claim 4, wherein the respiratory state determination unit is configured to calculate a first order equation representative of an approximate line for the XY-coordinate points for the respective respiratory states, and determine the state of the respiratory function of the subject on the basis of at least one of a slope and an X-intercept of the first order equation.

6. The apparatus of claim 1, wherein the different inspiratory volumes are set by mechanically limiting an inspiratory flow with use of a limiting device.

7. The apparatus of claim 1, wherein the different inspiratory volumes are set with use of a display device which displays the different inspiratory volumes.

8. The apparatus of claim 1, wherein the respiratory state detection unit is configured to apply calibration to each of the intrapleural pressures to calculate an absolute value of the intrapleural pressure.

9. The apparatus of claim 1, wherein the respiratory state detection unit is configured to estimate each of the intrapleural pressures from the pulse wave of the subject.

10. The apparatus of claim 9, wherein the pulse wave of the subject is measured in a non-invasive manner.

11. A non-transitory computer-readable storage medium encoded with a computer program, the program comprising instructions that, when executed by a data processing apparatus, define an respiratory function testing apparatus for testing a respiratory function of a subject, the apparatus comprising:
a respiratory state detection unit configured to acquire a first signal representative of different inspiratory volumes corresponding to a plurality of breaths of the subject and a second signal representative of intrapleural pressures corresponding to the respective different inspiratory volumes, and detect a plurality of respiratory states corresponding to the different inspiratory volumes and their corresponding intrapleural pressures: and
a respiratory state determination unit configured to capture a state of the respiratory function of the subject on the basis of the plurality of respiratory states corresponding to the different inspiratory volumes and their corresponding intrapleural pressures.
